(19) United States
(12) Patent Application Publication
(10) Pub. No.: US 2006/0008778 A1
(43) Pub. Date: Jan. 12, 2006

(54) Cosmetic enhancement device

(76) Inventor: Cynthia Spaulding Rosenberg, St. Catharines (CA)

Correspondence Address: BOYLE FREDRICKSON NEWHOLM STEIN & GRATZ, S.C., 250 E. WISCONSIN AVENUE, SUITE 1030, MILWAUKEE, WI, 53202, US

(21) Appl. No.: 11/133,105

(22) Filed: May 19, 2005

Related U.S. Application Data
(60) Provisional application No. 60/572,502, filed on May 20, 2004.
(60) Provisional application No. 60/653,087, filed on Feb. 16, 2005.

(51) Int. Cl. A61B 5/117

(52) U.S. Cl. 433/229

(57) ABSTRACT

A cosmetic enhancement device for enhancement of at least one of at least a portion of a lip area and at least a portion of a perioral area of a wearer. The device comprises an engaging member for placement between a gum and musculature of the at least one of the at least a portion of the lip area and the at least a portion of the perioral area of the wearer; and a fastener for maintaining the engaging member in place between the gum and the musculature of the at least one of the at least a portion of the lip area and the at least a portion of the perioral area of the wearer. In a further embodiment, the device comprises an engaging member for placement between a gum and musculature of the at least one of the at least a portion of the lip area and the at least a portion of the perioral area of the wearer, wherein the device is biasable to a closed position to maintain the engaging member in place between the gum and the musculature of the at least one of the at least a portion of the lip area and the at least a portion of the perioral area of the wearer.

### CROSS-REFERENCE TO RELATED APPLICATIONS

This applications claims priority from U.S. Provisional Patent Application Ser. Nos. 60/572,502, filed on May 20, 2004 and 60/653,087 filed Feb. 16, 2005, the entirety of which is incorporated by reference herein.

### FIELD OF THE INVENTION

The present invention relates in general to cosmetic enhancements relating to the lip area and/or perioral area. More particularly, the present invention relates to a cosmetic enhancement device.

### BACKGROUND OF THE INVENTION

Since about the 1960's, plastic surgeons have been practicing perioral enhancements and lip enhancement, also known as lip enlargement or lip augmentation. It has become much more popular recently, in part, due to the media attention that has been given to supermodels, movie stars, and every day people (e.g. reality TV) having such procedural enhancements performed.

The desire for lip and/or perioral enhancements range from slight and modest to extreme. Men and women of all ages are seeking such enhancements and reasons range from age-related, where people want a more youthful appearance, to reasons relating to enhancement of their natural features. For example, people are seeking lip enhancements because they simply have small or thin lips and desire a more pouty appearance with more fullness. In addition, as people age, muscle tension and tone weaken in the perioral area (e.g. cheek area and/or the area around the lip) and lip area, where the perioral area becomes more gaunt and the lip area begins to hang, wrinkle, lengthen and thin, consequently, enhancements are sought for these areas. Moreover, people appearing gaunt as a result of missing teeth may also seek enhancement in the perioral area and/or lip area.

In other cases, teeth can influence the posture of lips and cause lips to droop or hang. In such cases where teeth are particularly crooked or if there is an overbite or underbite, a plastic surgeon may first want oral surgery performed prior to lip enhancement, which adds further burden and cost to the patient.

Currently, the spectrum of procedures for enlargement of the lips ranges from an office procedure with no anesthesia and some swelling, bruising and discomfort, to a hospital procedure under general anesthesia, significant swelling, bruising, pain, and weeks off work.

Presently, there are three general procedures that are used for lip enhancement: injections of materials, such as collagen; grafting in substances such as gortex or fat; and local flaps and incisions that redistribute the tissues from inside the mouth to the outside. The results of the procedures can be temporary or permanent. More extreme measures are taken to enhance the perioral area (e.g. cheek area and/or the area around the lip) to achieve a fuller appearance.

All of these procedures have advantages and disadvantages, different types of anesthesia, recovery periods, pain and time off work. Like any surgery, such procedures have their risks. For instance, there may be numbness, swelling, ulcerations, scarring, unnatural movement when smiling or talking, and disfigurement, as a result of cosmetic enhancement procedures.

It is apparent, therefore, that there is a need for a way of achieving cosmetic enhancement in a more simple manner, that lacks adverse effects, is less costly and is completely reversible. There is also a need for noninvasive cosmetic enhancement.

### SUMMARY OF THE INVENTION

In one aspect of the present invention, there is provided a cosmetic enhancement device for enhancement of at least one of at least a portion of a lip area and at least a portion of a perioral area of a wearer, the device comprising:
an engaging member for placement between a gum and musculature of the at least one of the at least a portion of the lip area and the at least a portion of the perioral area of the wearer; and
a fastener for maintaining the engaging member in place between the gum and the musculature of the at least one of the at least a portion of the lip area and the at least a portion of the perioral area of the wearer.

In another aspect of the present invention, there is provided a cosmetic enhancement device for enhancement of at least one of at least a portion of a lip area and at least a portion of a perioral area of a wearer, the device comprising:
an engaging member for placement between a gum and musculature of the at least one of the at least a portion of the lip area and the at least a portion of the perioral area of the wearer, wherein the device is biasable to a closed position to maintain the engaging member in place between the gum and the musculature of the at least one of the at least a portion of the lip area and the at least a portion of the perioral area of the wearer.

In further aspects of the present invention, there is provided a kit for making the cosmetic enhancement device of the present invention.

The novel features of the present invention will become apparent to those of skill in the art upon examination of the following detailed description of the invention. It should be understood, however, that the detailed description of the invention and the specific examples presented, while indicating certain embodiments of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

Preferred embodiments of the present invention will now be described more fully with reference to the accompanying drawings, wherein like numerals denote like parts:

FIG. 1A shows a perspective view of a cosmetic enhancement device of a first embodiment of the present invention;

FIG. 1B shows a side elevational view of the jaws of a wearer showing two mounted cosmetic enhancement devices of FIG. 1A;

FIG. 2 shows an occlusal view of the upper and lower arches of a typical mouth of a wearer;

FIG. 3A shows a perspective view of a cosmetic enhancement device of another embodiment of the present invention;

FIG. 3B shows a side elevational view of the jaws of a wearer showing the mounted cosmetic enhancement device of FIG. 3A;

FIG. 4A shows a perspective view of a cosmetic enhancement device of a further embodiment of the present invention;

FIG. 4B shows a side elevational view of the jaws of a wearer showing the mounted cosmetic enhancement device of FIG. 4A;

FIG. 5A shows a perspective view of a cosmetic enhancement device of another embodiment of the present invention;

FIG. 5B shows a side elevational view of the jaws of a wearer showing the mounted cosmetic enhancement device of FIG. 5A;

FIG. 6A shows a perspective view of a cosmetic enhancement device of a further embodiment of the present invention in an open and closed position;

FIG. 6B shows a side elevational view of the jaws of a wearer showing the mounted cosmetic enhancement device of FIG. 6A;

FIG. 7A shows a perspective view of a cosmetic enhancement device of another embodiment of the present invention; and

FIG. 7B shows a side elevational view of the jaws of a wearer showing the mounted cosmetic enhancement device of FIG. 7A.

### DETAILED DESCRIPTION OF THE PREFERRED EMBODIMENTS

The present invention relates to a cosmetic enhancement device. The device, when placed in the mouth, pushes at least a portion of the lip area of the wearer outwardly to give the appearance of larger, poutier lip(s) and/or pushes at least a portion of the perioral area of the wearer outwardly to give the appearance of a fuller, less wrinkled perioral area.

The term “perioral” used herein is understood to include at least a portion of the cheek area and/or at least a portion of the area around the lip. The term “teeth” used herein is not only understood to include natural teeth but also dental work (e.g. dentures, caps, etc.) as well.

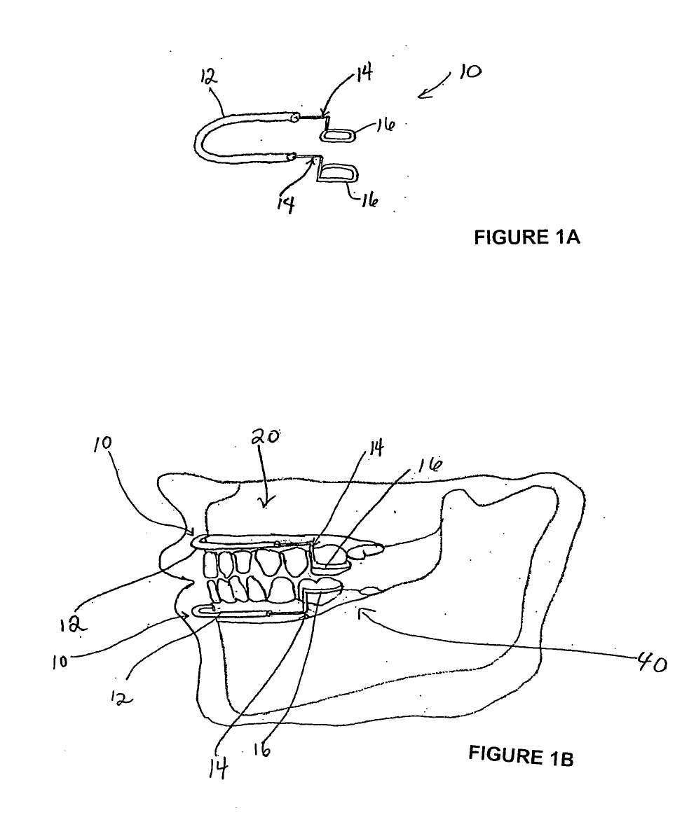

A first embodiment of the cosmetic enhancement device of the present invention is shown in FIGS. 1A and 1B and indicated generally by the numeral 10. The cosmetic enhancement device 10 is shown engaging an upper arch 20 and a lower arch 40. It will be appreciated that although the oral device 10 is shown on both the upper arch 20 and the lower arch 40, the device of the present embodiment may be used on one of the upper arch 20 and the lower arch 40 for enhancement. Moreover, it is understood that the device of the present invention, in general, may be used on the upper and/or lower arches.

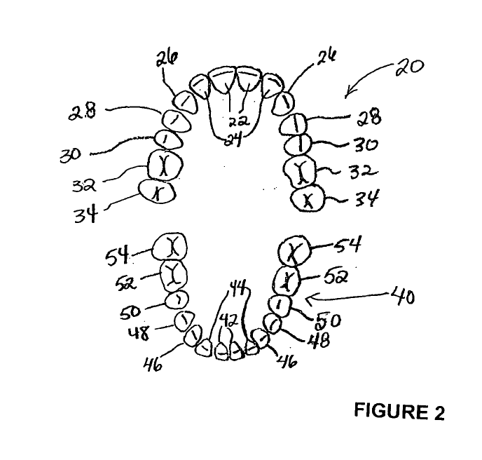

The cosmetic enhancement device 10 has an engaging member 12 disposed between the gum and the musculature of the lip area and perioral area. It is understood that the term “gum” used herein may also include the gum-line of the wearer. The engaging member 12 is generally U-shaped to follow the general gum-line of the anterior teeth. The engaging member 12 is made of a polymer material that is comfortable for the wearer. In this embodiment, the anterior teeth are considered to be from bicuspid to bicuspid, and the teeth distally of the cuspids are considered to be the posterior teeth, which are the molars. FIG. 2 shows the various teeth labeled for ease of reference. The upper arch 20 includes centrals 22, laterals 24, cuspids 26, first bicuspids 28, second bicuspids 30, first molars 32, and second molars 34. Similarly, the lower arch 40 includes opposing teeth that are defined as centrals 42, laterals 44, cuspids 46, first bicuspids 48, second bicuspids 50, first molars 52, and second molars 54. It will be understood that this is a typical arrangement of teeth and that in some people certain teeth are missing or may have been extracted. Therefore, it is understood that the device of the present invention can be configured to accommodate such situations. The device can also be configured for dentures.

The device 10 has a fastener 14, coupled to the engaging member 12, which is configured to engage the second molars (e.g. second molars 34 of the upper arch 20 and second molars 54 of the lower arch 40) to maintain the device 10 in place. The fastener 14 is a metal wire with one end of the metal wire coupled to the engaging member 12. The metal wire extends from the engaging member 12 into a loop 16. The loop 16 is shaped to engage the second molars to maintain the device 10 in place such that the engaging member 12 is generally aligned at about and/or above the gum-line of the anterior teeth (with respect to the upper arch 20) and at about and/or below the gum-line of the anterior teeth (with respect to the lower arch 40) when mounted in the wearer's mouth. As a result, the lip musculature is moved outwardly to give the appearance of a fuller lip and to lessen the appearance of wrinkles around the lip area. A portion of the engaging member 12 that follows the general gum-line of the anterior teeth (e.g. generally aligned at about and/or above the gum-line of the anterior teeth (with respect to the upper arch 20) and at about and/or below the gum-line of the anterior teeth (with respect to the lower arch 40)) also engages the underside of the cheek area. Consequently, this portion can also push the cheek area outwardly and give the appearance of a fuller cheek area (e.g. less gaunt) depending on the diameter of the engaging member 12.

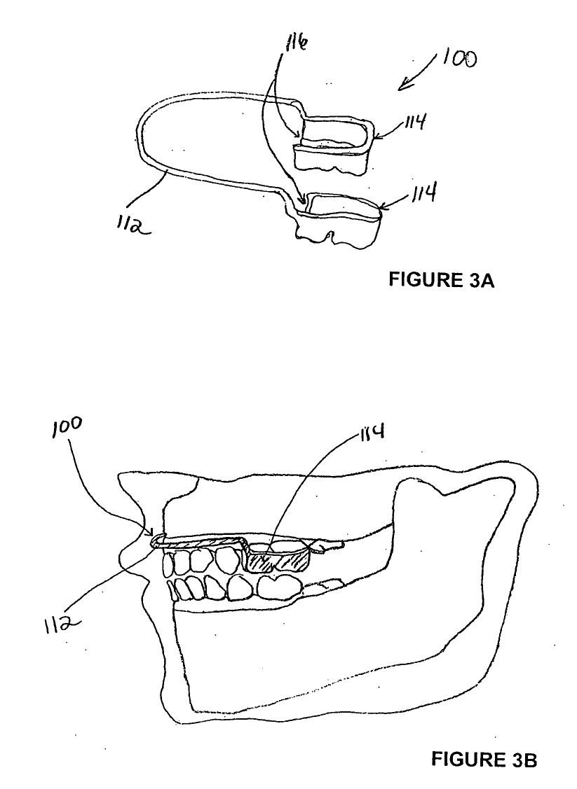

Another embodiment of the cosmetic enhancement device of the present invention is shown in FIGS. 3A and 3B and indicated generally by the numeral 100. The cosmetic enhancement device 100 has an engaging member 112 disposed between the gum and the musculature of the lip area and perioral area. The engaging member 112 is generally U-shaped to follow the general gum-line of the anterior teeth.

A fastener 114, integral with the engaging member 112, is configured to engage the first and second molars 32, 34 to maintain the device 100 in place. The fastener 114 is a mold of the first and second molars 32, 34, wherein one end of the mold has an opening 116 for allowing the wearer to slide the mold more easily over the molars and engage the teeth. The fastener 114 maintains the device 100 in place such that the engaging member 112 is generally aligned at about and/or above the gum-line of the anterior teeth when mounted in the wearer's mouth. As a result, the lip musculature is moved outwardly to give the appearance of a fuller lip and to lessen the appearance of wrinkles around the lip area. A portion of the engaging member 112 that follows the general gum-line of the anterior teeth (e.g. generally aligned at about and/or above the gum-line of the anterior teeth) also engages the underside of the cheek area. Consequently, this portion can also push the cheek area outwardly and give the appearance of a fuller cheek area (e.g. less gaunt) depending on the diameter of the engaging member 112.

The engaging member 112 and the fastener 114 are made of a polymer material that is comfortable in the mouth and rigid enough to maintain the device 100 in place.

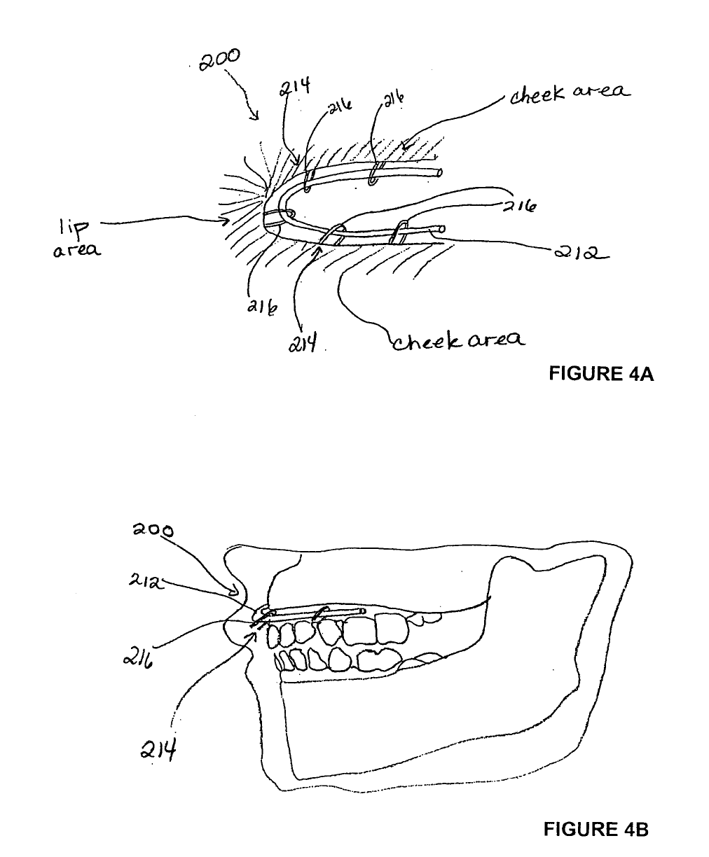

A further embodiment of the cosmetic enhancement device of the present invention is shown in FIGS. 4A and 4B and indicated generally by the numeral 200. The cosmetic enhancement device 200 has an engaging member 212 disposed between the gum and the musculature of the lip area and the perioral area. The engaging member 212 is generally U-shaped to follow the general gum-line of the anterior teeth.

A fastener 214 is attached to the underside of the cheek area and lip area. The fastener 214 is a number of loops 216 for threading the engaging member therethrough. The loops 216 maintain the engaging member 212 in place such that the engaging member 212 is generally aligned at about and/or above the gum-line of the anterior teeth when mounted in the wearer's mouth. As a result, the lip musculature is moved outwardly to give the appearance of a fuller lip and to lessen the appearance of wrinkles around the lip area. A portion of the engaging member 212 that follows the general gum-line of the anterior teeth (e.g. generally aligned at about and/or above the gum-line of the anterior teeth) also engages the underside of the cheek area. Consequently, this portion can also push the cheek area outwardly and give the appearance of a fuller cheek area (e.g. less gaunt) depending on the diameter of the engaging member 212.

The engaging member 212 is made of a polymer material that is comfortable in the mouth and the loops 216 are made of a suitable surgical material that is comfortable in the mouth and strong enough to maintain the device 200 in place.

Alternative embodiments and variations of the cosmetic enhancement device are also possible.

The engaging member may be made of any suitable material that is comfortable for the wearer. Such materials include, but are not limited to, polymers, such as silicones or waxes, and metals, such as stainless steel. The engaging member may be made from clear or pigmented material to match the pigment of the gum, in order to camouflage it.

Although the above-described embodiments have the engaging member disposed between the gum and the musculature of the lip area and perioral area. It is understood that the engaging member can be between the gum and the musculature of at least a portion of the lip area and/or at least a portion of the perioral area. Although the above-described embodiments have the engaging member configured to be generally aligned at about and/or above the gum-line of the anterior teeth and the anterior teeth are considered to be from bicuspid to bicuspid, the engaging member may extend more or less along the upper and/or lower arch depending on the amount of enhancement desired, depending on the area of enhancement desired (e.g., lip area and/or perioral area), and the comfort level of the wearer. For example, but not to be limited thereto, the engaging member may extend from cuspid to cuspid to enhance at least a portion of the lip area, may extend from molar to molar to enhance both at least a portion of the lip area and at least a portion of the cheek area, or may extend simply from lateral tooth to lateral tooth to enhance at least a portion of the lip area. The engaging member may also extend from beyond the molars on one side to beyond the molars on the other side of the arch. Also, if a wearer has uneven lips or uneven cheeks (e.g. one cheek is more sunken), the engaging member may extend asymmetrically, for instance, from a cuspid to a lateral tooth. Consequently, the engaging member, although described as generally U-shaped in the above embodiments, may be any suitable shape that can be configured to follow the general gum-line of the teeth. For example, a generally curve-shaped may be used.

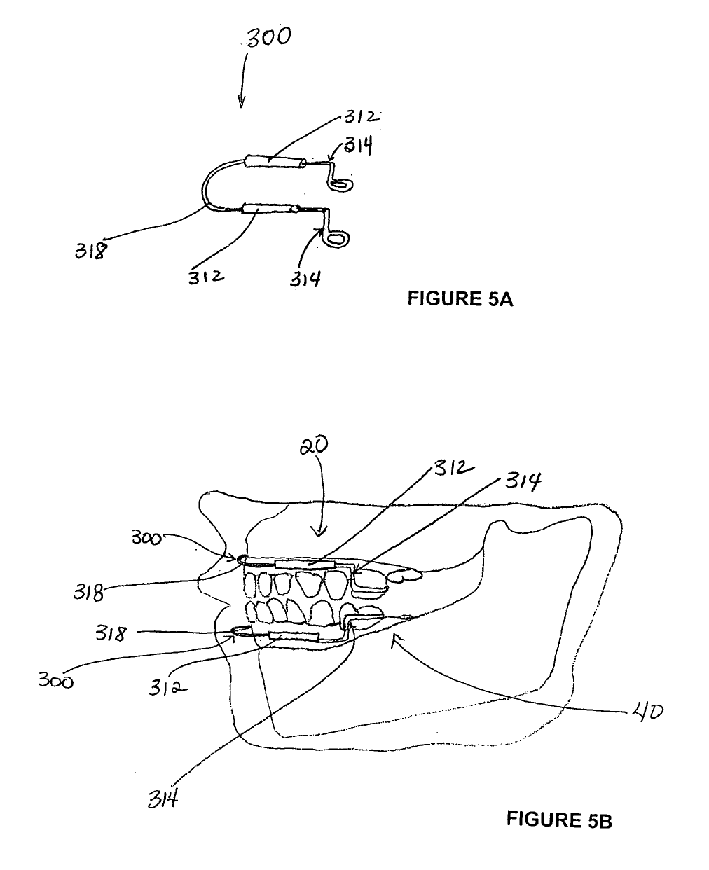

In a further embodiment, the device may also comprise more than one engaging member. For instance, as shown in FIGS. 5A and 5B, the device indicated generally by 300 has two linear engaging members 312, wherein a portion of the cheek areas are enhanced only. The engaging members 312 are disposed between the gum and the musculature of the cheek area. The engaging members are joined by a coupler 318, such as a metal wire, and the fastener 314 is similar to that for the embodiment described in FIGS. 1A and 1B. All of the appropriate variations discussed herein with respect to the engaging member and the fastener are applicable for this embodiment as well.

The actual size of the various engaging member(s) of the present invention may be varied depending on the specific enhancement desired. Therefore, the engaging member may be customized accordingly. Although it is typical for the cross-section of the engaging member to be generally circular, the cross-section may be any suitable shape for enhancement. For instance, the engaging member may be a flange that protrudes outwardly, inwardly, or angled in suitable directions. For example, the cross-section may appear more oval than circular. The cross-section may also appear flattened. The diameter of the cross-section of the engaging member may also be varied depending on the specific enhancement desired. For instance, the diameter can be large for more moderate to extreme enhancements or smaller for less enhancement. The diameter may also be varied along the length of the engaging member. The diameter may, for instance, be larger in the frontal portion of the engaging member to enhance the lip area and be smaller in the back portion (over the molars) of the engaging member to maintain the cheeks in their substantially natural shape or vice versa. The diameter of the engaging member may or may not be the controlling factor of enhancement. For example, the spacing between the engaging member and the gum may gauge the enhancement.

The engaging members of the present invention may be any suitable shape in order to achieve enhancement of at least a portion of the lip area and/or at least a portion of the perioral area. In other embodiments, the engaging member may be generally linear. For example, there may be two or more engaging members (e.g. FIGS. 5A and 5B) that may or may not be fastened to the teeth. The engaging members may be fastened to the teeth using the fasteners discussed herein and/or the engaging members may be held in place using a fastener, such as an adhesive for adhering to at least a portion of the gum, to the underside of at least a portion of the cheek area (e.g. perioral area), and/or to the underside of at least a portion of the lip area, to hold the device in place. For example, two engaging members may be used, wherein each engaging member is placed in between the cheek area and the gum area to enhance the cheek.

In other embodiments, there may be two or more devices on one arch of a wearer that may or may not be fastened to the teeth. The devices may be fastened to the teeth using the fasteners discussed herein and/or the engaging members may be held in place using a fastener, such as an adhesive for adhering to at least a portion of the gum, to the underside of at least a portion of the cheek area (e.g. perioral area), and/or to the underside of at least a portion of the lip area, to hold the device in place. For example, two devices may be used, wherein each device is placed in between the cheek area and the gum area, wherein the engaging member enhances the cheek.

The fasteners used in the present invention may be made of any suitable material that is comfortable for the wearer but strong enough to maintain the cosmetic enhancement device in place. Such materials include, but are not limited to, polymers, such as silicones or waxes, metals, such as stainless steel, and surgical material. The fasteners may also be any suitable fastener that maintains the device in place. For example, the fastener may be any type of tooth-engaging fastener. In one of the aforementioned embodiments, although wire is shaped to engage the second molars to maintain the device in place, the wire may simply be cemented to a tooth, similar to the procedure used for braces, to maintain the device in place. In addition, the fasteners may engage any tooth to maintain the device in place, however, it is of course more advantageous to engage teeth that are not as visible when the person smiles. Along these same lines, the fasteners may be clear or pigmented to match the tooth, in order to camouflage it from the viewer.

The fastener may also be any suitable adhesive to maintain the device in place. For example, in FIG. 3A, adhesive, acting as an additional fastener, may be added to the fastener 114 to further maintain the cosmetic enhancement device in place. Therefore, the adhesive can assist in providing additional support to other fasteners. Alternatively, the adhesive may be used on its own as a fastener without the aid of any other fastener. Adhesive may be applied to the device, placed in the mouth and adhered to at least a portion of the gum, at least a portion of teeth, to the underside of at least a portion of the cheek area, and/or to the underside of at least a portion of the lip area. In addition, or alternatively, the adhesive may first be applied to at least a portion of the gum, at least a portion of teeth, to the underside of at least a portion of the cheek area, and/or to the underside of at least a portion of the lip area and the device placed in the mouth to couple with the applied adhesive.

The adhesive used in the present invention may be any suitable adhesive for holding the device in place. The adhesive, for example, may be any suitable adhesive gel or powder such as that used by denture wearers.

Other types of fasteners may also include suction cups. The suction cups may be integral and/or coupled to the device. The suction cups can engage at least a portion of the gum, at least a portion of teeth, the underside of at least a portion of the cheek area, and/or the underside of at least a portion of the lip area to maintain the device in place. Adhesive may also be used in conjunction with the suction cups.

The loops 214 described in one of the aforementioned embodiments may be replaced with any type of suitable fastener to maintain the engaging member in place. The fastener may be attached to the underside of at least a portion of the perioral area and/or at least a portion of the lip area or attached to the gum. The fastener may also be attached quite close to the lip itself.

The device of the present invention may be configured for dentures and for teeth having a variety of dental work done thereto. Therefore, the device is suitable for many people desiring such enhancement.

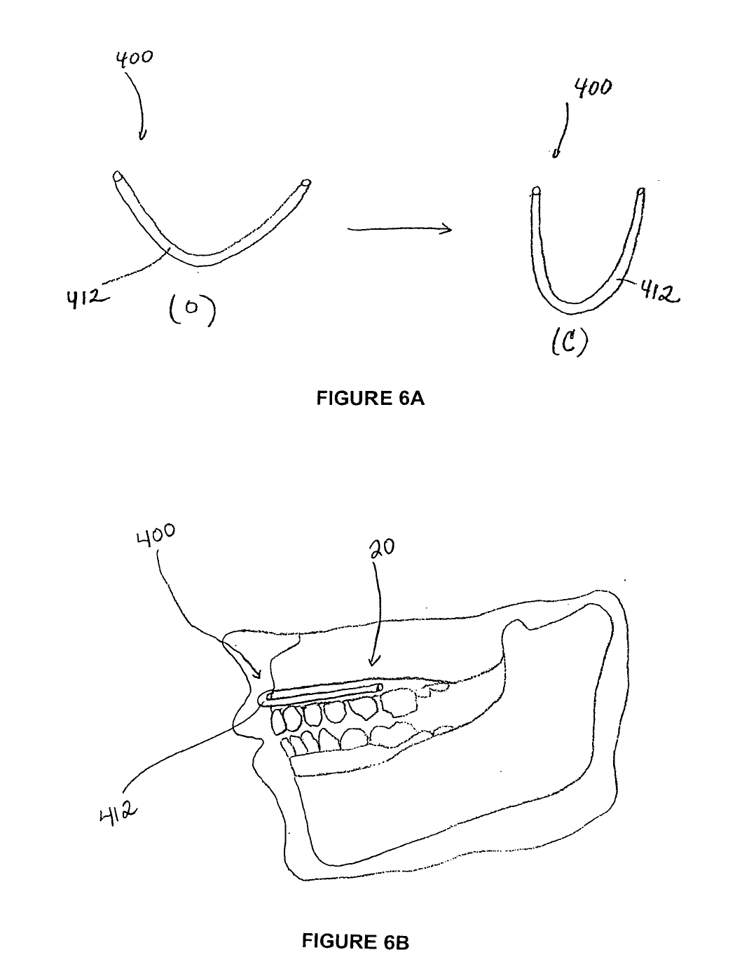

In other embodiments of the cosmetic enhancement device of the present invention, the cosmetic enhancement device does not need a fastener per se. For instance, the cosmetic enhancement device has an engaging member similar to that described for the aforementioned embodiments; generally U-shaped to follow the general gum-line of the anterior teeth. The engaging member is, however, biasable from an opened position for placement in the mouth to a closed position where it conforms to the general gum-line of the anterior teeth and is held in place. One embodiment of such a device is shown in FIGS. 6A and 6B. The device is indicated generally by the numeral 400 and has an engaging member 412. The engaging member 412 is made of a biasable material so that it may be opened, indicated as (O) in FIG. 6A, and placed in the wearer's mouth, and closed, indicated as (C) in FIG. 6A, to conform to the general gum-line of the anterior teeth so that it remains at about the gum-line of the anterior teeth and/or above the gum-line of the anterior teeth, as shown in FIGS. 6A and 6B. The biasable material may be, but not limited to, a bendable metal. Adhesive may also be applied to the device, placed in the mouth and adhered to at least a portion of the gum, at least a portion of teeth, to the underside of at least a portion of the cheek area, and/or to the underside of at least a portion of the lip area. All of the appropriate variations discussed herein with respect to the engaging member, are applicable to these embodiments as well. This device can be used to enhance at least a portion of the lip area and/or at least a portion of the perioral area. For example, and without being limited thereto, the device 300 of FIGS. 5A and 5B, is configured not to have the fastener 314. Therefore, the device would have two linear engaging members 312, wherein a portion of the cheek areas are enhanced only. The engaging members 312 are disposed between the gum and the musculature of the cheek area. The engaging members are joined by a coupler 318, such as the metal wire, which is biasable, as similarly described above with respect to FIGS. 6A and 6B, so that the device is able to conform to the general gum-line of the anterior teeth so that it remains at about the gum-line of the anterior teeth and/or above the gum-line of the anterior teeth (for the upper arch 20). Similarly for the lower arch 40, so that the device remains at about the gum-line of the anterior teeth and/or below the gum-line of the anterior teeth.

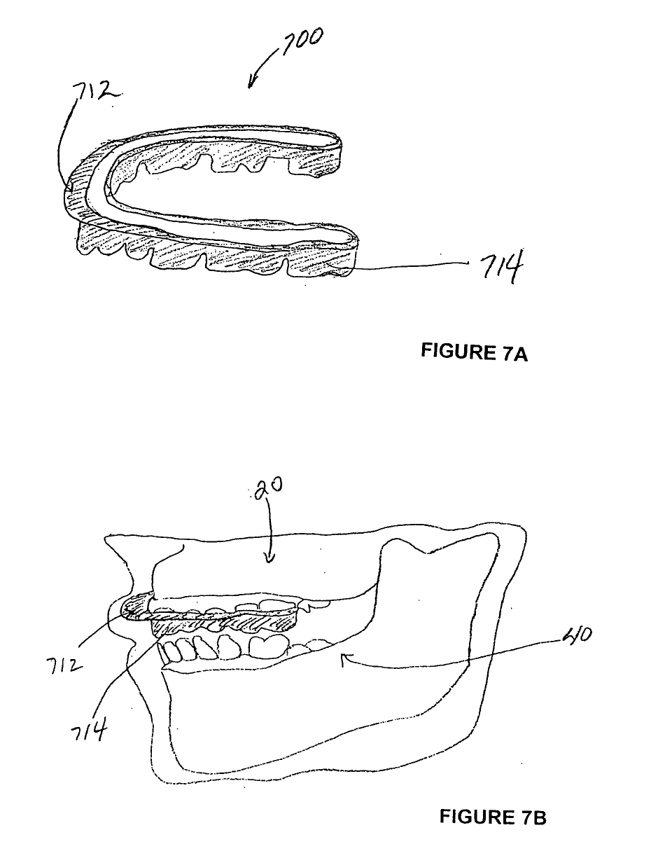

Another embodiment of the cosmetic enhancement device of the present invention is shown in FIGS. 7A and 7B and indicated generally by the numeral 700. The cosmetic enhancement device 700 has an engaging member 712 disposed between the gum and the musculature of the lip area. However, the engaging member may be configured to also be disposed between the gum and the musculature of the perioral area or may be configured to be disposed only between the gum and the musculature of the perioral area. The engaging member 712 is generally U-shaped to follow the general gum-line of the anterior teeth.

A fastener 714, integral with the engaging member 712, is configured to engage the teeth to maintain the device 700 in place. The fastener 714 is a mold of the teeth. The fastener 714 maintains the device 700 in place such that the engaging member 712 is generally aligned at about and/or above the gum-line of the anterior teeth when mounted in the wearer's mouth. An adhesive, as described above, may also be used as an additional fastener, to maintain the device in place. The device 700 may be made of any suitable material such as a polymer material that is comfortable in the mouth and rigid enough to maintain the device 700 in place. In addition, the material used may be such that the teeth of the wearer appear more appealing. For example, the device 700 may be made of any clear, shiny polymeric material that will give the appearance of polished, shiny teeth. The device may also be made from a material that has a suitable tooth colour. For instance, if the wearer has discoloured teeth, the fastener 714 may be made of a coloured/white material that can camouflage the wearer's discoloured teeth. All of the appropriate variations described above with respect to the other engaging members and the fasteners are applicable to these embodiments as well.

The device of the present invention may also comprise a safety catch to ensure that the device does not accidentally ‘pop-out’ of the wearer's mouth. The safety catch can simply be an additional fastener included with the device. For example, the device of FIG. 4a may include an additional fastener such as a wire leading from the engaging member 212 that fastens to a tooth of the wearer so that if the loops 216 break, the engaging member is still maintained in the wearer's mouth by the additional fastener.

The cosmetic enhancement device of the present invention may be made using a number of orthodontic methods known to those skilled in the art. The wearer may also be able to make the cosmetic enhancement device using a kit. For example, a kit may be provided that includes parts for the cosmetic enhancement device and instructions for assembly and use of the cosmetic enhancement device.

In an embodiment, the kit may comprise a moldable material to form a mold of at least a portion of the cosmetic enhancement device of the present invention; and instructions for assembly and use of the cosmetic enhancement device. The kit may also comprise a receptacle for receipt of the moldable material.

In a more specific embodiment, the kit comprises a receptacle; a moldable material for placement in the receptacle to apply to a cast of an impression of the teeth and at least a portion of the gum area of the wearer to form a mold of the cast, wherein the mold is configured to the cosmetic enhancement device; and instructions for assembly and use of the cosmetic enhancement device.

In another embodiment, the kit comprises a receptacle; a first moldable material for placement in the receptacle to make an impression of the teeth and at least a portion of the gum area of the wearer; a casting material to make a cast of the impression of the teeth and the at least a portion of the gum area of the wearer; a second moldable material for placement in the receptacle to apply to the cast to form a mold of the cast, wherein the mold is configured to the cosmetic enhancement device; and instructions for assembly and use of the cosmetic enhancement device.

In a typical embodiment, the kit includes a tray and a moldable material (e.g. rubber, silicone, or alginates) for placement in the tray to make an impression of the wearer's teeth and gum area (upper and/or lower arches). The tray is similar to a standard tray that is used for making teeth impressions. The tray is, however, deeper than most standard trays so that it extends from the top of the gum and around the teeth with respect to the upper arch, or from the bottom of the gum and around the teeth with respect to the lower arch. The tray may also be longer to extend past the teeth, such as past the molars. This type of tray ensures that a proper impression of the wearer's teeth and gum area is taken. The kit also includes a suitable material (e.g. plaster) to make a cast of the teeth impression and gum area. Therefore, this material is added to the tray containing the teeth impression and a cast made of the teeth and gum area. The resulting cast/model of the teeth and gum area is used to craft the cosmetic enhancement device. The kit also includes a suitable material (e.g. silicone, rubber, or alginate) for making the device. This material is applied to the cast/model of the teeth to form a suitable mold of the teeth and gum area. The amount of material used may be varied depending on the amount of enhancement desired between the gum and the musculature of at least a portion of the lip area and/or at least a portion of the perioral area. The mold is removed from the cast. The mold of the wearer's teeth and gum area is then used to make the cosmetic enhancement device. If the mold is of the upper arch, the mold includes a portion at about the gum-line and/or above the gum-line of the teeth and if the mold is of the lower arch, the mold includes a portion at about the gum-line and/or below the gum-line of the teeth. The mold may be left or simply cut to provide a device similar to the embodiment shown in FIGS. 3A and 3B and 7A and 7B, respectively. For the device of FIGS. 3A and 3B, the mold is cut to remove the mold of the anterior teeth to leave the portion of the mold that is above the gum-line of the upper arch. With respect to the posterior teeth, the opening 116 is simply cut out, yielding a device similar to the device 100. The device 100 can then be placed in the wearer's mouth to provide the desired enhancement. Various other molds may be used to achieve the cosmetic enhancement device of the present invention. For example, the mold may be a simple U-shaped mold, wherein the moldable material is placed therein to yield the engaging member of the device. Various fasteners, such as the loop 16 of device 10, may be then coupled to the lip engaging member.

In another embodiment, the kit includes a self molding heat formed mold, which may be heated by external heat, body heat and/or other suitable method.

In any of the kit embodiments of the present invention where molds are made, flanges, bulges, ridges and other protrusions of various materials may then be added to and/or integral with the mold to produce the desired cosmetic enhancement.

The device of the present invention may be used in a less invasive matter, compared to the procedures currently used by plastic surgeons, by using the fasteners, such as the loops made of the surgical material described above. The device of the present invention may also be used in a completely noninvasive manner as described herein, offering risk-free cosmetic enhancement, more particularly, lip and/or perioral enhancement.

The device of the present invention can be customized to fit any type of mouth. The degree of enhancement can be varied depending on the wearer's desire for mild to moderate to extreme enhancement for upper and/or lower arches of the mouth.

The embodiments and examples set forth herein are presented to best explain the present invention and its practical application and to thereby enable those skilled in the art to make and utilize the invention. Those skilled in the art, however, will recognize that the description and examples are presented for the purpose of illustration and example only. Other variations and modifications of the present invention will be apparent to those of skill in the art, and it is the intent of the appended claims that such variations and modifications be covered.

The description as set forth is not intended to be exhaustive or to limit the scope of the invention. Many modifications and variations are possible in light of the above teaching without departing from the spirit and scope of the following claims. It is contemplated that the use of the present invention can involve components having different characteristics. It is intended that the scope of the present invention be defined by the claims appended hereto, giving full cognizance to equivalents in all respects.

1. A cosmetic enhancement device for enhancement of at least one of at least a portion of a lip area and at least a portion of a perioral area of a wearer, the device comprising:
an engaging member for placement between a gum and musculature of said at least one of said at least a portion of the lip area and said at least a portion of the perioral area of the wearer; and
a fastener for maintaining the engaging member in place between the gum and the musculature of said at least one of said at least a portion of the lip area and said at least a portion of the perioral area of the wearer.

2. The device of claim 1, wherein the fastener is coupled to the engaging member.

3. The device of claim 1, wherein the fastener is integral with the engaging member.

4. The device of claim 1, wherein the engaging member is generally aligned at about the gum when mounted in a mouth of the wearer.

5. The device of claim 4, wherein the engaging member is generally aligned at about a gum-line of the teeth of the wearer.

6. The device of claim 1, wherein the engaging member is generally curve-shaped.

7. The device of claim 1, wherein the engaging member is generally U-shaped.

8. The device of claim 1, wherein the engaging member is generally linear.

9. The device of claim 1, wherein the engaging member has a generally circular cross-section or an oval cross-section.

10. The device of claim 1, wherein the fastener is loops attached to at least one of the underside of at least a portion of the lip area and at least a portion of the perioral area of the wearer.

11. The device of claim 1, wherein the fastener is at least one of a teeth-engaging fastener, an adhesive, a suction cup, and a mold of the teeth of the wearer.

12. The device of claim 11, wherein the fastener is the teeth engaging fastener.

13. The device of claim 11, wherein the fastener is the adhesive.

14. The device of claim 11, wherein the fastener is the mold of the wearer's teeth.

15. The device of claim 1, wherein the engaging member is for placement between the gum and musculature of said at least a portion of the lip area.

16. The device of claim 1, wherein the engaging member is for placement between the gum and musculature of said at least a portion of the perioral area.

17. The device of claim 1, wherein the device comprises at least two of said engaging members.

18. The device of claim 17, wherein the device further comprises at least one coupler for joining said engaging members.

19. The device of claim 1 further comprises a safety catch.

20. A cosmetic enhancement device for enhancement of at least one of at least a portion of a lip area and at least a portion of a perioral area of a wearer, the device comprising:
an engaging member for placement between a gum and musculature of said at least one of said at least a portion of the lip area and said at least a portion of the perioral area of the wearer, wherein the device is biasable to a closed position to maintain the engaging member in place between the gum and the musculature of said at least one of said at least a portion of the lip area and said at least a portion of the perioral area of the wearer.

21. The device of claim 20, wherein the engaging member is biasable to a closed position.

22. The device of claim 20, wherein the engaging member is generally aligned at about the gum when mounted in a mouth of the wearer.

23. The device of claim 22, wherein the engaging member is generally aligned at about a gum-line of the teeth of the wearer.

24. The device of claim 20, wherein the engaging member is generally curve-shaped.

25. The device of claim 20, wherein the engaging member is generally U-shaped.

26. The device of claim 20, wherein the engaging member is generally linear.

27. The device of claim 20, wherein the engaging member has a generally circular cross-section or an oval cross-section.

28. The device of claim 20, wherein the engaging member is for placement between the gum and musculature of said at least a portion of the lip area.

29. The device of claim 20, wherein the engaging member is for placement between the gum and musculature of said at least a portion of the perioral area.

30. The device of claim 20, wherein the device comprises at least two of said engaging members.

31. The device of claim 30, wherein the device further comprises at least one coupler for joining said engaging members.

32. A kit for the cosmetic enhancement device of claim 1, wherein the kit comprises:
a moldable material to form a mold of at least a portion of the cosmetic enhancement device; and
instructions for assembly and use of the cosmetic enhancement device.

33. A kit for the cosmetic enhancement device of claim 19, wherein the kit comprises:
a moldable material for placement in the receptacle to form a mold of at least a portion of the cosmetic enhancement device; and
instructions for assembly and use of the cosmetic enhancement device.

34. The kit of claim 32 further comprises a receptacle for receipt of the moldable material.

35. The kit of claim 33 further comprises a receptacle for receipt of the moldable material.

36. A kit for making the cosmetic enhancement device of claim 1, wherein the kit comprises:
a receptacle;
a moldable material for placement in the receptacle to apply to a cast of an impression of the teeth and at least a portion of the gum area of the wearer to form a mold of the cast, wherein the mold is configured to the cosmetic enhancement device; and
instructions for assembly and use of the cosmetic enhancement device.

37. A kit for making the cosmetic enhancement device of claim 1, wherein the kit comprises:
a receptacle;
a first moldable material for placement in the receptacle to make an impression of the teeth and at least a portion of the gum area of the wearer;
a casting material to make a cast of the impression of the teeth and said at least a portion of the gum area of the wearer;
a second moldable material for placement in the receptacle to apply to the cast to form a mold of the cast, wherein the mold is configured to the cosmetic enhancement device; and
instructions for assembly and use of the cosmetic enhancement device.

38. The kit of claim 37, wherein at least two of the first moldable material, the casting material, and the second moldable material are the same material.
